(19) United States
(12) Plant Patent Application Publication
(10) Pub. No.: US 2015/0195970 P1
(43) Pub. Date: Jul. 9, 2015

(54) Pear tree named 'PremP027'

(50) ;

(71) Applicant: Allan G. White, Havelock North (NZ)

(72) Inventor: Allan G. White, Havelock North (NZ)

(21) Appl. No.: 14/544,401

(22) Filed: Dec. 31, 2014

Related U.S. Application Data
(60) Provisional application No. 61/924,158, filed on Jan. 6, 2014.

(51) Int. Cl. A01H 5/00

(52) U.S. Cl. PLT/176

(57) ABSTRACT

A new and distinct pear tree named ‘PremP027’ is disclosed. The new pear is notable for its attractive appearance, superb texture, improved flavor and long shelf life.

### LATIN NAME OF THE GENUS AND SPECIES OF THE PLANT CLAIMED

Pyrus bretschneideri×Pyrus communis

### VARIETY DENOMINATION

‘PremP027’

### BRIEF DESCRIPTION OF THE PHOTOGRAPHS

FIG. 1 shows the fruit of a ‘PremP027’ pear tree

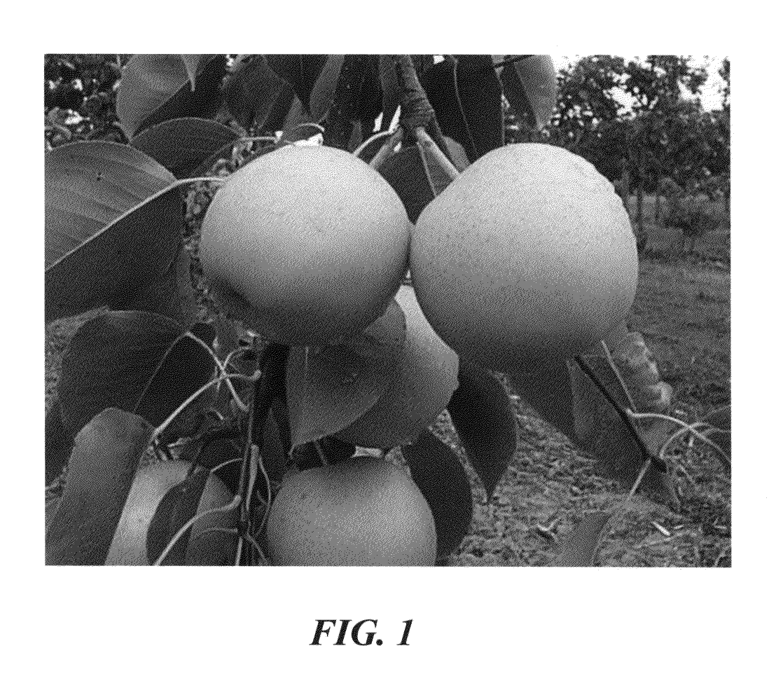

### BRIEF DESCRIPTION OF THE VARIETY

The new variety of pear tree ‘PremP027’ was created in the course of a planned breeding program carried out at Motueka, New Zealand. ‘PremP027’ originated as a result of a controlled cross of ‘Max Red Bartlett’ (seed parent, U.S. Plant Pat. No. 741) and ‘Snowflake’ (pollen parent, not patented) in 1989. ‘PremP027’ was selected in 1996 as a single plant from among a population of seedlings derived from the parents, and was selected for its quality of fruit. It is distinguished from its parents by its shape and color. ‘Max Red Bartlett’ fruit is pyriform in shape with high coverage of red skin color compared to ‘PremP27’ which has no red skin color. ‘PremP27’ is almost round with slight tapering at the stem and calyx end. Fruit of ‘PremP27’ ripens 7-10 days later than ‘Max red Bartlett’. Fruit of ‘PremP27’ has less prominent lenticels than ‘Snowflake’ and ripens approximately 14-21 days earlier. ‘PremP27’ is also distinguishable from other known varieties, such as ‘Nijisseiki’(U.S. Plant Pat. No. 8,529). ‘Nijisseiki’ has more round fruit than ‘PremP27’ and ripens approximately 7-10 days earlier. Asexual propagation by budding was first carried out in Motueka and Havelock North, New Zealand. The variety has since been observed over a number of asexually propagated generations, and has been found to remain true to type.

### DETAILED BOTANICAL DESCRIPTION OF THE VARIETY

The following detailed botanical description is based on observations made during the 2013 growing season at Parker, Wash. on 5 year old trees. All colors are described according to The Royal Horticultural Society Colour Chart as published in 2001. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Vigor — Medium; Habit — Upright; Size — Height 10 feet; Spread 5 feet; Trunk — Diameter at graft union 10.2 cm; Bark texture — smooth.
Branch: Size — Diameter 3.5 cm at 86 cm above ground; Crotch angle relative to trunk — 70°.
Dormant one-year-old shoot: Size — diameter 1.7 cm, length 86 cm; Color — 146B; Internode length — 6-8 cm; Lenticels — quantity 10 per square inch, color 155B, diameter 0.05 cm.
Flowers: Petal — Quantity per flower 5; Bloom period — First bloom April 12, full bloom April 18 in Parker, Wash.
Leaf: Attitude in relation to shoot — upward; Blade margin — serulate; Size — length 8.5 cm, width 7 cm; Color — upper surface 146D; Color of lower surface — 145C.
Petiole: Length — 5 cm; Diameter — 0.1 cm Fruit: Size — diameter 9.6 cm, length 15 cm; Ribbing absent; Size of eye 2 cm; Depth of eye basin 0.5 cm; Width of eye basin 1 cm; Stalk — length 4.5 cm, width at limb attachment 0.5 cm, color N176D; Depth of stalk cavity 0.25 cm; Width of stalk cavity 1 cm; Size of lenticels 0.1 cm; Color of lenticels 200D; Ground color of skin 154C;
Quantity per cluster 2 — 3; Flesh is very crisp and very juicy; Flesh color 155B; Quantity of seeds 10; Width of seed 0.5 cm; Length of seed 1 cm; Seed color Black 200A.
Disease resistance/susceptibility: None noted.
Storage: Excellent storage performance of at least 3 months.
Use: Fresh Market.

1. We claim a new and distinct pear tree substantially as shown and described herein.
